(19) United States
(12) Patent Application Publication
(10) Pub. No.: US 2024/0252468 A1
(43) Pub. Date: Aug. 1, 2024

(54) TREATMENT OF TUNNELING WOUNDS USING METRONIDAZOLE-CONTAINING COMPOSITIONS

(71) Applicant: S.L.A. PHARMA AG, Liestal (CH)

(72) Inventors: Justin SLAGEL, Hertfordshire (GB); David LUBOWSKI, Hurtsville, NSW (AU)

(21) Appl. No.: 18/561,852

(22) PCT Filed: Jan. 21, 2022
(86) PCT No.: PCT/IB2022/000025

§ 371 Date: Nov. 17, 2023

Related U.S. Application Data
(60) Provisional application No. 63/139,857, filed on Jan. 21, 2021.

(51) Int. Cl. A61K 31/4164; A61K 9/00; A61K 45/06; A61K 47/06

(52) U.S. Cl. CPC A61K 31/4164; A61K 9/0014; A61K 45/06; A61K 47/06

(57) ABSTRACT

A method of treating tunneling wounds comprising administering a topical composition containing metronidazole into a passageway of the tunneling wound. One advantage of the composition is that topical administration of metronidazole results in a primarily local effect, and thus, side effects observed from systemic administration are avoided.

### CROSS REFERENCE TO RELATED APPLICATIONS

This application claims priority to U.S. Provisional Patent Application No. 63/139,857 filed on Jan. 21, 2021 in the name of David LUBOWSKI and Justin SLAGEL and entitled “Treatment of Tunneling Wounds Using Metronidazole-Containing Compositions,” which is hereby incorporated by reference herein in its entirety.

### FIELD OF THE INVENTION

The present invention relates to the treatment of tunneling wounds using a composition comprising metronidazole or a pharmacologically acceptable salt thereof.

### BACKGROUND OF THE INVENTION

When treating wounds, occasionally wounds are encountered that are best described as “tunneling” wounds. A tunneling wound is substantially tunnel-shaped, having an opening at the surface and extending into the flesh, forming a tunnel that is usually longer, and sometimes substantially longer, than the width of the opening. Tunneling wounds can occur by themselves or in combination with a larger open wound, which may include one or more tunneling wounds. Because of their depth, tunneling wounds can present significant risks of infection and can be particularly difficult to heal completely. Thus, it is desirable to provide treatment that removes wound exudates (which can support bacterial growth) and promotes healing of a tunneling wound, including the deepest portions of the wound. Poor healing in the deepest portions of a wound can lead to abscesses if the shallower portion of the wound heals first and closes off the tunnel.

### SUMMARY OF THE INVENTION

It is an object of the present invention to use a composition comprising metronidazole to treat tunneling wounds and other fistula-like structures.

In one aspect, a method of treating tunneling wounds is described, said method comprising administering a therapeutically effective amount of a topical composition into a passageway of a tunneling wound of a subject, wherein said topical composition comprises from about 5% to 25% of metronidazole or pharmaceutically acceptable salt thereof, and a pharmaceutically acceptable excipient or carrier.

In another aspect, a kit for the treatment of tunneling wounds is described, wherein the kit includes a container that holds a topical composition as described herein, a catheter or tube, and a syringe wherein both the container and the catheter/tube can be separately mated with the syringe.

These and other advantages and features of the present invention will be described more fully in a detailed description of the preferred embodiments which follows.

### BRIEF DESCRIPTION OF THE DRAWINGS

FIG. 1 is an image of an example of the tunneling wound.

FIG. 2 is an image of the insertion of the catheter or tube into the passageway of the tunneling wound.

FIG. 3 is an image of a catheter or other tube “locked” to the syringe.

### DETAILED DESCRIPTION OF THE INVENTION

Throughout the instant specification and claims, the following definitions and general statements are applicable.

As used herein, whether in a transitional phrase or in the body of a claim, the terms “comprise(s)” and “comprising” are to be interpreted as having an open-ended meaning. That is, the terms are to be interpreted synonymously with the phrases “containing at least,” “having at least” or “including at least.” When used in the context of a process, the term “comprising” means that the process includes at least the recited steps but may include additional steps. When used in the context of a composition, the term “comprising” means that the composition includes at least the recited features or components but may also include additional features or components.

The terms “consists essentially of” or “consisting essentially of” have a partially closed meaning, that is, they do not permit inclusion of steps or features or components which would substantially change the essential characteristics of a process or composition; for example, steps or features or components which would significantly interfere with the desired properties of the compositions described herein, i.e., the process or composition is limited to the specified steps or materials and those which do not materially affect the basic and novel characteristics of the invention.

The terms “consists of” and “consisting of” are closed terminology and allow only for the inclusion of the recited steps or features or components.

As used herein, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 10%.

As used herein, “treating” means reducing, hindering or inhibiting the development of, controlling, alleviating and/or reversing the symptoms in the individual to which a combination or composition described herein has been administered, as compared to the symptoms of an individual not being treated as described. A practitioner will appreciate that the combinations, compositions, dosage forms and methods described herein are to be used in concomitance with continuous clinical evaluations by a skilled practitioner to determine subsequent therapy.

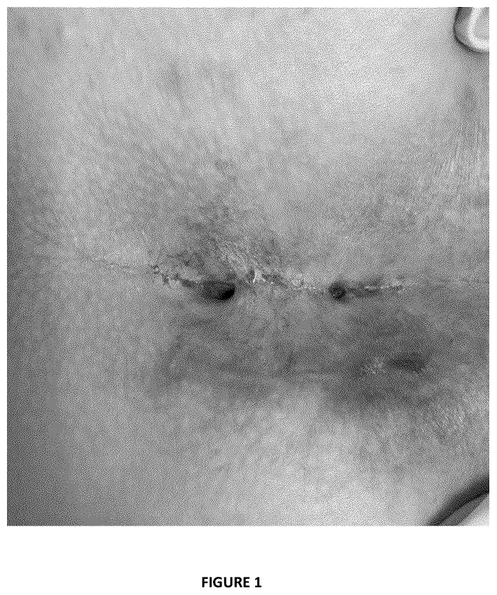

As defined herein, a “tunneling wound,” also known as a “tracking wound,” is a wound that has a passageway underneath the skin surface, with or without an opening or passageway (e.g., a wound edge). The tunnel can be shallow or deep, can be long or short, can be branched or unbranched. There are many causes associated with the formation of the “tunneling wound” including, but not limited to, an infection, debris as a result of incomplete cleansing, the presence of a foreign object such as gauze, an abscess, excess fluid, a wound in the midline of the body, a wound edge closed prematurely, and a wound which heals in such a way as to allow full epithelialization and closure of parts of the wound while leaving one or more openings extending down into the deeper unhealed part of the wound. In one embodiment, the tunneling wound comprises a passageway under the central epithelialized area which hasn't healed, for example, a midline wound (see, e.g., FIG. 1). For the purposes of the instant application, the tunneling wound is intended to include a sinus tract, a fistula, a fistula-like wound, a pilonidal sinus wound, hidradenitis, or other open wound that can only be accessed with a topical composition using the method described herein. It should be appreciated that there are certain risk factors, such as diabetes, which increase the likelihood of a tunneling wound as described herein.

“Substantially devoid” is defined herein to mean that none of the indicated substances are intentionally added to or present in the composition.

For the purposes of this application, a “surgical operation” includes, but is not limited to, a hemorrhoidectomy, a fistulotomy, a fissurectomy, a sphincterotomy, sphincteroplasty, a perineal incision after abdominoperineal resection, incision and drainage of an abscess, and excision of pilonidal abscess or sinus.

Metronidazole (or “Flagyl”), having the formula 2-methyl-5-nitroimidazole-1-ethanol, is a nitroimidazole derivative with activity against anaerobic protozoa, aerobic and microaerophilic bacteria. Metronidazole also possesses anti-inflammatory properties, which are less well understood.

Disadvantageously, in its oral and intravenous forms, metronidazole is frequently associated with a number of serious side effects. These negative side effects include gastrointestinal manifestations, such as nausea, vomiting, a metallic taste in the mouth, or inflammation of the oral cavity. Serious neurological side effects can occur which usually manifest as numbness or tingling of the extremities. These neurological side effects can be debilitating, are often irreversible, and necessitate the cessation of the metronidazole. Serious hematological, cardiovascular, or renal complications are also common and can be life-threatening. In addition, the overgrowth of opportunistic organisms such as Candida can result from oral or intravenous metronidazole treatment. Oral metronidazole is also known to interact in an adverse manner with other medications, such as oral anticoagulants (e.g. coumadin), which can cause potentially fatal bleeding.

Topical metronidazole has previously been used for a number of skin conditions (e.g., rosacea) or as a topical vaginal preparation in the treatment of vaginal infections (e.g. trichomonas). However, these preparations are contained in a medium containing alcohol, which would result in stinging and burning when used on damaged skin or exposed tissue in a wound.

The present inventors have previously introduced a topical composition that overcomes the shortcomings of the prior art, said topical composition to be discussed hereinbelow. The present inventors used the topical composition to treat external tissue damage, for example, burns, Bartholin's cysts, pruritus ani/perianal folliculitis, decubitus ulcers, perianal/sacral ulcers, skin abrasions, lacerations, cellulitis, superficial thrombophlebitis, pyoderma gangrenosum, and erythema nodosum. In each case, the topical composition was intended to be topically applied and rubbed by hand over the entire area, until the composition disappears.

Without wishing to be bound by any particular theory, it is believed that the use of a topical composition comprising metronidazole, directly applied to the diseased or otherwise affected region, is primarily a local effect. Minimal systemic absorption is observed and therefore systemic side effects are effectively reduced or eliminated. As such, the dose of metronidazole can be altered for specific tissue and applied directly to the diseased or otherwise effected area thereby increasing the efficacy of the medication.

Broadly, the instant invention relates to a method of treating tunneling wounds using a topical composition comprising metronidazole or a pharmacologically acceptable salt thereof. In one embodiment, the method comprises topically administering into a tunneling wound of a subject, a therapeutically effective amount of a topical composition comprising from about 5% to 25% of metronidazole or pharmaceutically acceptable salt thereof, and a pharmaceutically acceptable excipient or carrier. The composition will be discussed in detail below.

In order to access the tunneling wound, a tube or catheter or cannula needs to be inserted into the wound edge opening so that the composition can be introduced into the passageway of the tunneling wound. In one embodiment, the topical composition is introduced into the passageway until the passageway has been substantially filled with topical composition. In order to accomplish this, the catheter or tube can be inserted into the passageway and the topical composition delivered as the catheter or tube is slowly withdrawn from the passageway. The introduction of the topical composition can cease once the topical composition is no longer accommodated in the passageway. As discussed hereinbelow, the topical composition has a viscosity that facilitates passage through the tube or catheter to the tunneling wound passageway as well as ensures that the applied topical composition remains in, e.g., does not easily flow out of, the tunneling wound passageway after introduction. Following introduction into the passageway of the tunneling wound, the wound edge opening can be covered with a dressing, e.g., gauze or some other sterile bandage.

Advantageously, the topical composition introduced into the passageway of the tunneling wound replaces the necessity for packing material in the tunneling wound because the tunneling wound passageway is substantially filled with the topical composition.

Prior to introduction of the topical composition to the passageway of the tunneling wound, the wound edge opening can be optionally debrided, if necessary, and the area cleaned. For example, in one embodiment, the passageway can be flushed and drained. If there is an abscess deep in the passageway, the abscess is preferably drained prior to introduction of the topical composition. The topical composition can be introduced once, twice, three, or more times per day, as necessary, and as understood by the person skilled in the art. In a preferred embodiment, gradual improvement will be noted with each successive application, with the method of treatment running until the tunneling wound no longer has a passageway and the risk of infection within said passageway has been mitigated, as readily determined by the person skilled in the art. The dose frequency will vary according to the age, body weight, and response of the patient.

Preferably, the tunneling wound is a substantially healthy granulating wound prior to introduction of the topical composition. That said, there are circumstances where the wound is not a substantially healthy granulating wound prior to the introduction of the topical composition, as understood by the person skilled in the art. If the wound is not considered a substantially healthy granulating wound, the wound may need to be cleaned, e.g., using a bottle brush to remove debris or excess granulation, prior to introduction of the topical composition thereto. Regardless, in a preferred embodiment, no surgical operations were performed at least 7 days, at least 14 days, at least 21 days, or at least 28 days prior to introduction of the topical composition to the passageway. In another embodiment, a minor surgical procedure can be performed to provide an opening to the passageway that permits the insertion of the tube or catheter therein, but otherwise, no surgical operations were performed at least 7 days, at least 14 days, at least 21 days, or at least 28 days prior to introduction of the topical composition to the passageway.

In a preferred embodiment, the method of treating tunneling wounds described herein does not require negative pressure wound therapy.

The topical compositions can of course be used to treat humans or other mammals, such as veterinary animals including, without limitation, dogs, cats, other household pets, horses, farm animals, and the like.

### Compositions

The topical compositions for use in the method described herein comprise, consist of, or consist essentially of, metronidazole dissolved or dispersed in a pharmaceutically acceptable excipient or carrier. According to one embodiment, the topical composition takes the form of a cream or an ointment having a viscosity that ensures delivery through a tube or catheter into the passageway of the tunneling wound. Further, it is important that the applied topical composition remains in, e.g., does not easily flow out of, the tunneling wound passageway after introduction. It should be appreciated by the person skilled in the art that “ensuring delivery through a tube or catheter” means that the composition will flow through the tube or catheter, whether the flow is induced by displacement instead of by gravity, capillary action, etc.

In one embodiment, the metronidazole-containing topical composition comprises, consists of, or consists essentially of, metronidazole or a pharmacologically acceptable salt thereof at a concentration of about 5 wt % to about 25 wt %, more preferably about 5 wt % to about 15 wt %, and most preferably about 10 wt %, in a pharmaceutically acceptable carrier. All concentrations are based on the total weight of the composition. In some embodiments, the metronidazole is the sole active agent. Notably, the topical composition used in the methods described herein are substantially devoid of at least one of collagen, gelatin, Hepar sulphuris calcareum or other similar profiled Calcarea or Sulphur salt or acid, Lachesis muta or other remedy with a similar profile, Mercurius Solubilis or similar mercury containing remedy, Silica or other silica containing compounds, ciprofloxin, methylcellulose, propylene glycol, polyethylene glycol, rifamycin and derivatives thereof, oxazolidinone, stem cells, and any combination thereof. Furthermore, the topical composition is not a glue and will not set or harden at some time subsequent to introduction into the passageway.

The topical compositions used in the methods described herein are generally creamy or viscous suspensions or ointments comprising, consisting of, or consisting essentially of, metronidazole and at least one additional component or pharmaceutically acceptable excipient or carrier including preservatives, chelating agents, surfactants, thickeners, thickeners-solubilizers, buffers, anti-microbials, co-solvents, or lubricants. Preferably, the topical composition does not include an alcohol because of the painful effect caused by alcohol on damaged skin. Further, the pH of the composition is preferably 5.5 to 8.5, and more preferably a pH of about 7.

Preservatives may include, but are not limited to, tetrasodium ethylene-diamine tetraacetic acid (EDTA), methylparaben, benzophenone-4, methylchloroisothiazolinone, sodium benzoatemethylisothiazolinone, and the like, and mixtures thereof. Preservatives, when used, are typically present in an amount from about 0.01% to 10% weight, preferably about 0.05% to 4% weight, and more preferably, from about 0.1% to 2% weight, based on the total weight of the composition. Preferred moisturizers include, but are not limited to, wheat protein (e.g., laurdimonium hydroxypropyl hydrolyzed wheat protein), hair keratin amino acids, sodium peroxylinecarbolic acid, panthenol, tocopherol (Vitamin E), dimethicone, arachidylglucoside and the like, and mixtures thereof. Moisturizers, when used, are typically present in an amount from about 0.01% to 10% weight, preferably about 0.05% to 1.5% weight, more preferably, from about 0.1% to 1%, based on the total weight of the composition. Preferred surfactants, including both the foaming and non-foaming type, include, but are not limited to, sodium laureth sulfate, sodium laureth-13 carboxylate, disodium laureth sulfosuccinate, disodium cocoamphodiacetate, glycol stearate, PEG-150 distearate and the like, and mixtures thereof. More preferably, at least one amphoteric surfactant is included in the composition, selected from the group consisting of lauroamphocarboxypropionate, lauroamphopropionate, lauroamphoglycinate, lauroamphocarboxyglycinate, lauroamphopropylsulfonate, lauroamphocarboxypropionic acid, myristoamphocarboxy-propionate, myristoamphopropionate, myristoamphoglycinate, myristoamphocarboxyglycinate, myristoamphopropylsulfonate, myristoamphocarboxypropionic acid, cocoamphocarboxypropionate, cocoamphopropionate, cocoamphoglycinate, cocoamphocarboxyglycinate, cocoamphopropylsulfonate, cocoamphocarboxypropionic acid and mixtures thereof. Non-toxic non-ionic surfactants can also be included as wetting agents and dispersants. The surfactant component may be present in an amount from about 0.1% to about 20%, based on the total weight of the composition. An effective amount of a lubricant such as a natural or synthetic fat or oil, e.g., a tris-fatty acid glycerate or lecithin may be present.

Another category of functional ingredients within the metronidazole-containing topical compositions are thickeners. A thickener will usually be present in amounts anywhere from 0.1% to 20% by weight, preferably from about 0.5% to 10% by weight of the composition. Exemplary thickeners are cross-linked polyacrylate materials. Gums may be employed such as xanthan, carrageenan, karaya, pectin and locust beans gum. Under certain circumstances the thickening function may be accomplished by a material also serving as a silicone or emollient. For instance, silicone gums having a viscosity in excess of 10 mPas and esters such as glycerol stearate have dual functionality.

Alternatively, the topical composition is a lotion and the pharmaceutically acceptable excipient or carrier contains oils and emollients such as hydrocarbon oils and waxes, silicone oils, vegetable, animal or marine fats or oils, glyceride derivatives, fatty acids or fatty acid esters, lanolin and derivatives, wax esters, sterols, phospholipids and the like, as well as emulsifiers (nonionic, cationic, anionic, amphoteric), although some of the emollients inherently possess emulsifying properties. These same general ingredients can be formulated into a cream rather than a lotion, or into gels, by utilization of different proportions of the ingredients and/or by inclusion of thickening agents such as gums or other forms of hydrophilic colloids.

Emollients are often incorporated into the metronidazole-containing topical compositions. Levels of such emollients may range from about 0.5% to about 60%, preferably between about 5% and 30% by weight of the total composition. Emollients may be classified under such general chemical categories as esters, fatty acids and hydrocarbons. Esters may be mono- or di-esters. Acceptable examples of fatty di-esters include, but are not limited to, dibutyl adipate, diethyl sebacate, diisopropyl dimerate, and dioctyl succinate. Acceptable branched chain fatty esters include, but are not limited to, 2-ethyl-hexyl myristate, isopropyl stearate and isostearyl palmitate. Acceptable tribasic acid esters include, but are not limited to, triisopropyl trilinoleate and trilauryl citrate. Acceptable straight chain fatty esters include, but are not limited to, lauryl palmitate, myristyl lactate, oleyl eurcate and stearyl oleate.

Suitable fatty acids include those compounds having from 10 to 20 carbon atoms. Especially preferred are compounds such as cetyl, arachidyl, behenyl, cetearyl, myristyl, palmitic and stearyl acids.

Exemplary hydrocarbons which may serve as emollients are those having hydrocarbon chains anywhere from 12 to 30 carbon atoms. Specific examples include, but are not limited to, mineral oil, petroleum jelly, paraffin oil, squalene and isoparaffins.

Alternatively, the topical composition may be in the form of an oil, a suspension, a microemulsion, or vesicles.

In one particularly preferred embodiment, the pharmaceutically acceptable carrier is nonaqueous, comprising at least one hydrocarbon compound. Preferably, the carrier comprises a mixture of at least two semi-solid saturated hydrocarbon compounds. Suitable non-aqueous carriers include at least one of white petrolatum (USP) (also known as white soft paraffin (BP)), Vaseline™, Aquaphor (a combination of mineral oil, petrolatum ceresin and lanolin), lanolin, or a petroleum-based carrier.

In one embodiment, the metronidazole is applied in admixture with a dermatologically acceptable carrier (e.g., as a lotion, cream, ointment, soap or the like) so as to facilitate passage through a tube or catheter to the tunneling wound passageway as well as to ensure that the applied topical composition remains in, e.g., does not easily flow out of, the tunneling wound passageway after introduction.

Various types of other ingredients may be present in the metronidazole-containing topical compositions used in the methods described herein including, but not limited to, starches, sugars, microcrystalline cellulose, diluents, granulating agents, lubricants, surfactants, binders, disintegrating agents, and the like, as readily understood by the person skilled in the art.

Whatever the medium of delivery, the additional components or pharmaceutically acceptable carriers of the topical composition are preferably inert, ensuring no or negligible deactivation or oxidation of the metronidazole, as well as being generally incapable of causing any adverse effects on the skin areas to which it is applied.

At least one ingredient in the metronidazole-containing topical compositions can be a pharmaceutically acceptable salt, as understood by those of skill in the art. As used herein, by “pharmaceutically acceptable salt” it is meant those salts which are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art, such as hydrochloride, hydrobromide, mesylate, acetate, trifluoroacetate, propionate, fumarate, tartrate, citrate, phosphate, succinate, bisulfate, etc.

In one embodiment, the topical composition comprises metronidazole powder (USP) that has been mixed with white petrolatum (USP) and the mixture passed through a mixer known as an ointment mill to produce a 10 wt % metronidazole composition having a “fluffy” texture.

Actual dosage levels of active ingredients in the topical compositions may be varied so as to obtain an amount of the active compound(s) that is effective to achieve the desired response for a particular patient. It is within the skill of the art to start doses of the topical compound at levels lower than required to achieve the desired therapeutic effect and to gradually increase the dosage until the desired effect is achieved.

It should be appreciated by the skilled artisan that the topical composition may further comprise a therapeutic amount of at least one additional therapeutic agent to add to its effectiveness. Additional therapeutic agents that may be added include, but are not limited to, at least one of: calcium channel blockers, e.g., nifedipine and diltiazem; steroids, e.g., hydrocortisone or a pharmacologically acceptable derivative thereof; analgesic agents, preferably from the amide or ester class such as pramoxine or benzocaine; antimicrobial agents (antibacterial or antiviral), e.g., amoxicillin-clavulonic acid, erythromycin, tetracycline, clindamycin or doxycycline; substances that either promote skin integrity or inhibits skin breakdown, e.g., vitamin E, aloe, zinc oxide or other barrier cream; anti-inflammatory agents, e.g., a non-steroidal anti-inflammatory agent selected from aminosalicylic acid, ibuprofen, sulindac, piroxicam or diflunisal. Accordingly, in another embodiment, the metronidazole-containing topical composition comprises, consists of, or consists essentially of, metronidazole or a pharmacologically acceptable salt thereof at a concentration of about 5 wt % to about 25 wt %, more preferably about 5 wt % to about 15 wt %, and most preferably about 10 wt %, at least one additional therapeutic agent, and a pharmaceutically acceptable carrier. All concentrations are based on the total weight of the composition. In some embodiments, the metronidazole is the sole active agent. Notably, the topical composition used in the methods described herein are substantially devoid of at least one of collagen, gelatin, Hepar sulphuris calcareum or other similar profiled Calcarea or Sulphur salt or acid, Lachesis muta or other remedy with a similar profile, Mercurius Solubilis or similar mercury containing remedy, Silica or other silica containing compounds, ciprofloxin, methylcellulose, propylene glycol, polyethylene glycol, rifamycin and derivatives thereof, oxazolidinone, stem cells, and any combination thereof. Furthermore, the topical composition is not a glue and will not set or harden at some time subsequent to introduction into the passageway.

The metronidazole-containing topical compositions described herein can be formulated as a lotion having a viscosity of from 4,000 to 10,000 mPas, a fluid cream having a viscosity of from 10,000 to 20,000 mPas, or a cream or a gel having a viscosity of from 20,000 to 100,000 mPas or above. The final viscosity preferably facilitates passage through a tube or catheter to the tunneling wound passageway as well as to ensure that the applied topical composition remains in, e.g., does not easily flow out of, the tunneling wound passageway after introduction, as understood by the person skilled in the art.

Because of its ease of administration, a cream, lotion, gel or ointment represents the most advantageous topical composition dosage unit form, and such forms may be prepared to be inserted into, and remain in, the passageway of the tunneling wound.

In another aspect, the metronidazole-containing topical compositions described herein can be used to treat tunneling wounds, wherein a therapeutically effective amount of the metronidazole-containing topical compositions are administered into a passageway of a tunneling wound of a subject, wherein said topical composition comprises from about 5% to 25% of metronidazole or pharmaceutically acceptable salt thereof, and a pharmaceutically acceptable excipient or carrier.

### Kit

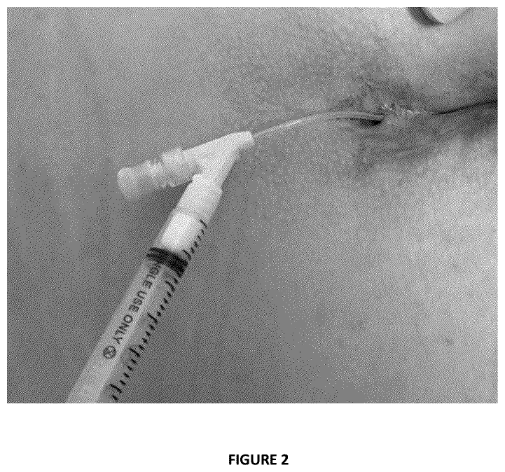

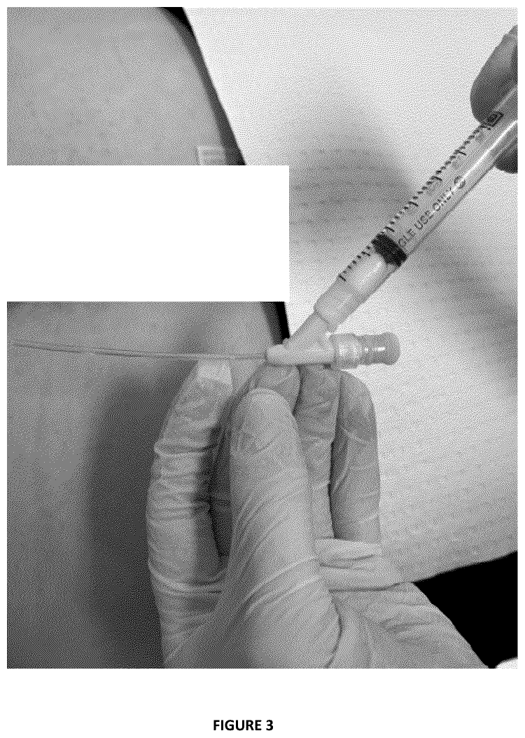

The metronidazole-containing topical composition can be packaged in a suitable container to suit its viscosity and intended use by the user. Preferably, the container can be mated with a syringe, for example using a luer lock mechanism, so that the topical composition can be easily drawn into the syringe. After introduction into the syringe, the syringe can be unlocked from the container and a catheter or other tube “locked” to the syringe (see, e.g., FIG. 3). Following insertion of the catheter or tube into the passageway of the tunneling wound, the topical composition can be introduced by engaging the plunger of the syringe to deliver the topical composition to the passageway (see, e.g., FIG. 2). It should be appreciated by the person skilled in the art that this is just one example of a container for the topical composition and other alternatives are readily understood by the person skilled in the art. It is not necessary to lock the container comprising the topical composition to the syringe and/or the catheter. In another embodiment, the container is a pre-filled single use container comprising an amount of topical composition contained therein and already having a tube or catheter attached thereto.

Accordingly, in another aspect, a kit for the treatment of tunneling wounds is described, wherein the kit includes a container that holds a topical composition as described herein, a catheter or tube, and a syringe wherein both the container and the catheter/tube can be separately mated with the syringe. For example, the mating mechanism can be a luer lock or the like, as understood by the person skilled in the art.

As introduced hereinabove, preferably, the tunneling wound is a substantially healthy granulating wound prior to introduction of the topical composition and no surgical operations were performed at least 7 days, at least 14 days, at least 21 days, or at least 28 days prior to introduction of the topical composition to the passageway. That said, there are circumstances where the wound is not a substantially healthy granulating wound prior to the introduction of the topical composition, as understood by the person skilled in the art. If the wound is not considered a substantially healthy granulating wound, the wound may need to be cleaned, e.g., using a bottle brush to remove debris or excess granulation, prior to introduction of the topical composition thereto. Accordingly, an embodiment of the kit for the treatment of tunneling wounds includes a container that holds a topical composition as described herein, a catheter or tube, a syringe, and a bottle brush, wherein both the container and the catheter/tube can be separately mated with the syringe. For example, the mating mechanism can be a luer lock or the like, as understood by the person skilled in the art. The bottle brush is included to remove excess granulation tissue so that the kit can be used to treat the tunneling wound in a simple office procedure. Further, the kit comprising the bottle brush can further comprise a local anaesthetic, such as an ampule comprising a single dose of ropivacaine or bupivacaine, to locally mitigate the pain associated with the cleaning of the wound with the bottle brush.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

1. A method of treating tunneling wounds comprising administering a therapeutically effective amount of a topical composition into a passageway of a tunneling wound of a subject, wherein said topical composition comprises from about 5% to 25% of metronidazole or pharmaceutically acceptable salt thereof, and a pharmaceutically acceptable excipient or carrier.

2. The method of claim 1, further comprising inserting a tube or catheter into a wound edge opening of the passageway of the tunneling wound, wherein the topical composition is introduced into the tunneling wound via the tube or catheter.

3. The method according to claim 2, wherein the topical composition is introduced into the tunneling wound until the passageway has been substantially filled with topical composition.

4. The method according to claim 2, wherein the tube or catheter is slowly withdrawn from the tunneling wound as topical composition is introduced to the tunneling wound.

5. The method of claim 1, wherein the topical composition is administered once, twice, three, or more times per day.

6. The method of claim 1, wherein no surgical operations were performed at least 7 days, at least 14 days, at least 21 days, or at least 28 days prior to introduction of the topical composition to the passageway.

7. The method of claim 1, wherein the pharmaceutically acceptable excipient or carrier is non-aqueous.

8. The method of claim 1, wherein the metronidazole concentration is about 10 wt %, based on the total weight of the composition.

9. The method of claim 7, wherein the non-aqueous carrier comprises at least one hydrocarbon compound.

10. The method of claim 9, wherein the at least one hydrocarbon compound comprises white petrolatum (USP).

11. The method of claim 1, wherein said the topical composition is in the form of an ointment, lotion, gel, foam or cream.

12. The method of claim 1, wherein the topical composition has a fluffy texture.

13. The method of claim 1, wherein the metronidazole is the sole active agent.

14. The method of claim 1, wherein the topical composition further comprises an additional therapeutic agent.

15. The method of claim 1, wherein the topical composition has a viscosity that facilitates passage through the tube or catheter while ensuring that the topical composition does not easily flow out of the tunneling wound after introduction.

16. The method of claim 1, wherein the topical composition is substantially devoid of at least one of collagen, gelatin, Hepar sulphuris calcareum or other similar profiled Calcarea or Sulphur salt or acid, Lachesis muta or other remedy with a similar profile, Mercurius Solubilis or similar mercury containing remedy, Silica or other silica containing compounds, ciprofloxin, methylcellulose, propylene glycol, polyethylene glycol, rifamycin and derivatives thereof, oxazolidinone, stem cells, and any combination thereof.

17. The method of claim 1, wherein the topical composition is not a glue and will not set or harden at some time subsequent to introduction into the passageway of the tunneling wound.
